(19) United States
(12) Patent Application Publication
(10) Pub. No.: US 2001/0016341 A1
(43) Pub. Date: Aug. 23, 2001

(54) Process for preparing gamma-polyglutamic acid from high-viscous culture broth

(76) Inventors: Ho-Nam Chang, Taejon (KR); Sang-Yup Lee, Taejon (KR); Jin-Hwan Do, Taejon (KR); Sun-Hoon Kwon, Taejon (KR)

Correspondence Address: KNOBBE, MARTENS, OLSON & BEAR, Sixteenth Floor, 620 Newport Center Drive, Newport Floor, CA, 92660-8016, US

(21) Appl. No.: 09/750,443

(22) Filed: Dec. 28, 2000

(30) Foreign Application Priority Data
Dec. 29, 1999 (KR) 1999-0064504

(51) Int. Cl. C12P 13/14; C12P 21/04; C12N 1/00; C12N 1/20

(52) U.S. Cl. 435/110; 435/71.3; 435/832; 435/839; 435/836

(57) ABSTRACT

The present invention relates to a process for preparing γ-polyglutamic acid (γ-PGA) from high-viscous culture broth, more particularly, to an economical and efficient process for preparing γ-polyglutamic acid from high-viscous culture broth with easy removal of microorganisms and a subsequent concentrating process employing a filtration membrane. The present invention provides the process for preparing γ-polyglutamic acid from high-viscous culture broth which comprises the steps of: culturing γ-polyglutamic acid-producing microorganism for 15-30 hours under condition of pH 5.0-7.5 and 30-40° C. to obtain high-viscous culture broth with a concentration of 20-30 g/L; removing microorganism from the high-viscous culture broth thus obtained by adjusting pH to 2-4 or 7-9 and centrifuging at 3,000-9,000 rpm for 10-50 minutes; and, obtaining γ-polyglutamic acid by concentrating the culture broth employing filter and precipitating γ-polyglutamic acid by addition of alcohol. In accordance with the invention, polyglutamic acid can be prepared from high-viscous culture broth in an economical and efficient manner, which would make it possible that polyglutamic acid can be applied widely as a universal material.

### BACKGROUND OF THE INVENTION

1. Field of the Invention

The present invention relates to a process for preparing γ-polyglutamic acid (“γ-PGA”) from high-viscous culture broth, more particularly, to an economical and efficient process for preparing γ-polyglutamic acid from high-viscous culture broth with easy removal of microorganisms and subsequent concentration employing a filtration membrane.

2. Background of the Invention

Polyglutamic acid produced by microorganism is completely biodegradable, thus, may be used as an ingredient of foodstuff, cosmetics, paint, oil remover, and surfactant in place of water-soluble non-degradable macromolecules. Recently, researches on the use of polyglutamic acid as medical material, functional carrier, membrane material, and electrical material are being actively made in the art(see: Kishida, A. and Murakami, K. et al., J. Bioactive and Compatible Polymers, 13:271-278, 1998; U.S. Pat. No. 5,693,751).

Although there have been many reports on the optimization of medium in batch culture (see: Ko, Y. K. and Gross, R. A., Biotechnol. Bioeng., 57:430-437, 1998) and on the supply of substrate in fed-batch culture (see: Korean patent laid-open publication No. 99-48816) for the production of highly concentrated polyglutamic acid, the method for isolation and recovery of polyglutamic acid from high-viscous culture broth still employs conventional high-speed centrifugation and alcohol-aided precipitation. Since the culture broth with polyglutamic acid concentration of 20-30 g/L is highly viscous, centrifugation for 30 minutes at 12,000 rpm is required to remove microorganisms by the conventional method (see: Japanese patent laid-open publication (Hei) 8-163993). In order to recover polyglutamic acid from the supernatant after centrifugation, alcohol precipitation method in which the volume of alcohol added is 2-4 times of that of polyglutamic acid solution is generally employed. However, the more volume of culture broth is treated, the more volume of alcohol is consumed, which brings about high-cost and environmental problems.

Under the circumstance, in order to widely use polyglutamic acid as a universal material, there are strong reasons for exploring and developing a process for preparing polyglutamic acid in an economical and efficient manner.

### SUMMARY OF THE INVENTION

The present inventors have made an effort to develop an economical and efficient process for preparing γ-polyglutamic acid from highly-viscous culture broth of γ-polyglutamic acid-producing microorganism, and finally found that: up to 60% of γ-polyglutamic acid produced by fermentation can be successfully recovered through the removal of microorganisms from culture broth by controlling pH of the broth and subsequent concentration employing filtration membrane, while reducing the volume of alcohol consumption for recovery of γ-polyglutamic acid to the level of ⅕ of that consumed by the conventional method.

A primary object of the present invention is, therefore, to provide an economical and efficient process for preparing γ-polyglutamic acid from high-viscous culture broth of γ-polyglutamic acid producing microorganism with easy removal of microorganisms and subsequent concentration employing filtration membrane.

### BRIEF DESCRIPTION OF THE DRAWINGS

The above and the other objects and features of the present invention will become apparent from the following descriptions given in conjunction with the accompanying drawings, in which:

FIG. 1 is a graph showing the change in viscosity depending on pH of the culture broth.

FIG. 2 is a graph showing the change in electric charge on cell surface.

FIG. 3 is a graph showing the concentrating efficiency of culture broth employing ultrafiltration membrane with exclusion molecular weight of 500 kDa.

### DETAILED DESCRIPTION OF THE INVENTION

The process for preparing γ-polyglutamic acid from high-viscous culture broth of the invention comprises the steps of: culturing γ-polyglutamic acid-producing microorganism for 15-30 hours under a condition of pH 5.0-7.5 and 30-40° C. to obtain high-viscous culture broth with a concentration of 20-30 g/L; removing microorganism from the high-viscous culture broth thus obtained by adjusting pH to 2-4 or 7-9 and centrifuging at 3,000-9,000 rpm for 10-50 minutes; and, obtaining γ-polyglutamic acid by concentrating the culture broth employing filter membrane and precipitating γ-polyglutamic acid by addition of alcohol.

The process for preparing γ-polyglutamic acid from high-viscous culture broth of the invention is illustrated in more detail in accordance with the following steps.

Step 1: Fermentation of a Microorganism

High-viscous culture broth is obtained by culturing γ-polyglutamic acid-producing microorganism, where Bacillus licheniformis (ATCC 9945A) or Bacillus subtilis, is preferably used as a microorganism used for producing γ-polyglutamic acid. High-viscous culture broth with concentration of 20-30 g/L can be obtained by fermentation performed in a culture medium containing 60-100 g/L glycerol, 10-30 g/L L-glutamic acid, 5-20 g/L citric acid, 3-10 g/L NH₄Cl, 0.1-1.0 g/L K₂HPO₄, 0.1-2.0 g/L MgSO₄.7H₂O, 0.01-0.1 g/L FeCl₃. 6H₂O, 0.05-0.5 g/L CaCl₂. 2H₂O and 0.05-0.5 g/L MnSO₄.H₂O for 15-30 hours under a condition of pH 5.0-7.5 and 30-40° C.

Step 2: Removal of Microorganisms from Culture Broth

Microorganisms are removed from the culture broth by controlling pH of high-viscous culture broth obtained in step 1 followed by centrifugation: the viscosity of the culture broth can be lowered by adjusting pH to 2-4 or 7-9, and then centrifugation is performed at 3,000-9,000 rpm for 10-50 minutes to remove microorganisms.

Step 3: Obtaining of γ-Polyglutamic Acid

γ-Polyglutamic acid can be obtained by concentrating the microorganism-free culture broth employing filter membrane and precipitating γ-polyglutamic acid by addition of alcohol: filtration is performed in order to reduce the volume of alcohol consumption required for recovery of γ-polyglutamic acid by precipitation, where an ultrafiltration membrane with exclusion molecular weight of 50-100 kDa or a filtration membrane with pore size of 0.15-0.5 μm may be preferably used, and ethanol is preferably added to the concentrate at a volume ratio of 4:1-2:1 to obtain γ-polyglutamic acid by precipitation.

The present invention is further illustrated in the following examples, which should not be taken to limit the scope of the invention.

### EXAMPLE 1

### Preparation of γ-Polyglutamic Acid

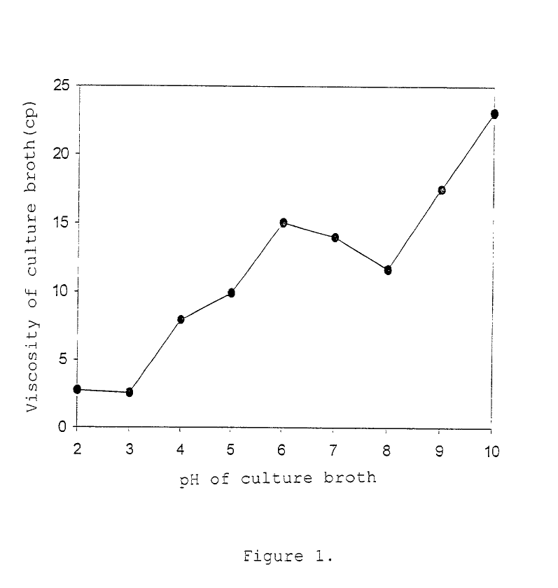

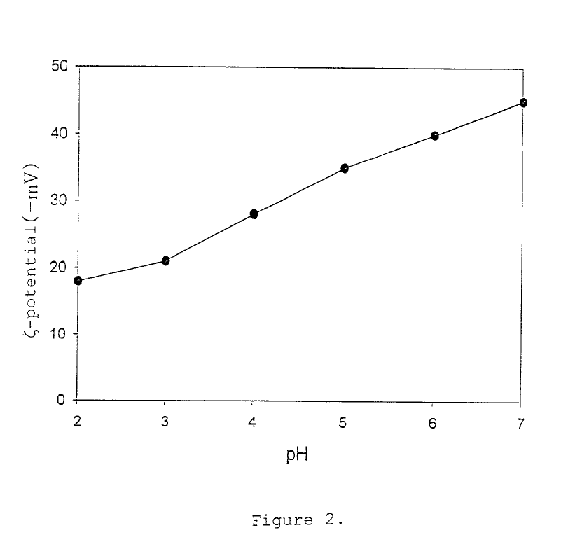

Bacillus licheniformis (ATCC 9945A) was inoculated into 5 L fermenter containing 3 L of culture medium comprising 80 g/L glycerol, 20 g/L L-glutamic acid, 12 g/L citric acid, 7g/L NH₄Cl, 0.5 g/L K₂HPO₄, 0.5 g/L MgSO₄. 7H₂O, 0.04 g/L FeCl₃. 6H₂O, 0.15 g/L CaCl₂. 2H₂O and 0.104 g/L MnSO₄.H₂O, and cultured under a condition of 37° C., pH 6.5 and a flow of mixture of pure oxygen and air to maintain dissolved oxygen tension of above 30% air saturation. After batch culture for 24 hours, high-viscous culture broth with γ-polyglutamic acid concentration of 20-25 g/L was obtained. In order to reduce the viscosity of culture broth which makes centrifugation difficult, pH of the culture broth was lowered, and the change in viscosity of the culture broth depending on pH change was monitored. FIG. 1 is a graph showing the change in viscosity depending on pH of the culture broth. As shown in FIG. 1, 6-fold reduction in viscosity of culture broth was attained by lowering pH to 3.0, meanwhile, adjusting pH to 1-2 caused degradation of microorganism. And, up to approximately pH 8.0, viscosity was shown to be decreased, however, viscosity began to increase above pH 8.0. FIG. 2 is a graph showing the change in electric charge on cell surface depending on pH. As lowering pH, the electric charge on the cell surface of microorganism decreased to ½, which renders centrifugation more effective by easier flocculation of microorganism. Since controlling of pH of the culture broth affected viscosity of culture broth and flocculation of microorganism, microorganisms were able to be effectively removed from the culture broth containing 20-30 g/L γ-polyglutamic acid by adjusting pH to 3 followed by centrifugation at 6,000 rpm for 30 minutes. The centrifugal force required for removal of microorganisms was reduced to ¼ of that required (12,000 rpm, 30 minutes) in prior art method disclosed in Japanese patent laid-open publication (Hei) 8-163993.

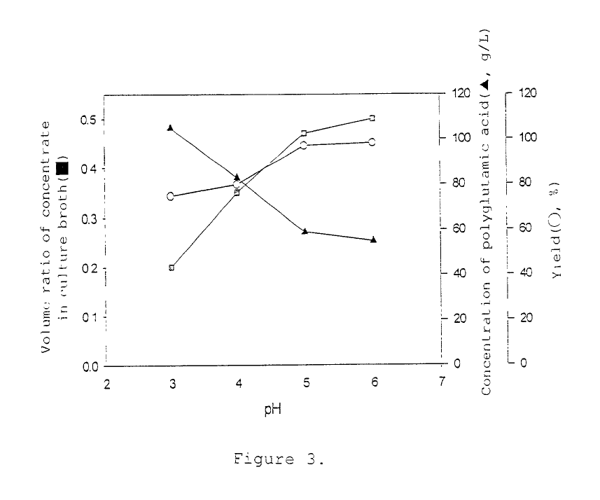

Since γ-polyglutamic acid is a linear macromolecule with a molecular weight of 1,000-2,000 kDa, the amount of alcohol consumption for recovery of γ-polyglutamic acid from the culture broth can be reduced by concentrating culture broth employing filtration membrane, thus, by using an ultrafiltration membrane with exclusion molecular weight of 50 kDa and a peristaltic pump, γ-polyglutamic acid concentration of 100 g/L was obtained (see: FIG. 3). FIG. 3 is a graph showing concentrating efficiency of culture broth employing an ultrafiltration membrane with exclusion molecular weight of 500 kDa. As shown in FIG. 3, 1 L of γ-polyglutamic acid concentrate with 100 g/L was obtained by concentrating 4 L of culture broth with 25 g/L. Sixty gram of γ-polyglutamic acid was recovered from the precipitate obtained by adding 2.5 L of ethanol to the concentrate above, thus, it was clearly demonstrated that the amount of ethanol consumption, in accordance with the invention, can be reduced to approximately ⅕ of the amount (12 L) of ethanol consumption by the conventional method in which filtration membrane-employed concentrating process is not included. Accordingly, the process for preparing γ-polyglutamic acid of the invention results in the reduction of cost, which would make it possible that γ-polyglutamic acid can be applied widely as a universal material.

As clearly illustrated and demonstrated as above, the present invention provides an economical and efficient process for preparing γ-polyglutamic acid from highly-viscous culture broth with easy removal of microorganisms by lowering viscosity through controlling of pH and subsequent concentration employing filtration membrane. In accordance with the invention, polyglutamic acid can be prepared from high-viscous culture broth in an economical and efficient manner, which would make it possible that polyglutamic acid can be applied widely as a universal material.

What is claimed is:

1. A process for preparing γ-polyglutamic acid from high-viscous culture broth which comprises:
(i) culturing γ-polyglutamic acid-producing microorganism for 15-30 hours under a condition of pH 5.0-7.5 and 30-40° C. to obtain high-viscous culture broth with a concentration of 20-30 g/L;
(ii) removing microorganism from the high-viscous culture broth thus obtained by adjusting pH to 2-4 or 7-9 and centrifuging at 3,000-9,000 rpm for 10-50 minutes; and,
(iii) obtaining γ-polyglutamic acid by concentrating the culture broth employing filter membrane and precipitating γ-polyglutamic acid by addition of alcohol.

2. The process for preparing γ-polyglutamic acid from high-viscous culture broth of claim 1, wherein the microorganism is Bacillus licheniformis or Bacillus subtilis.

3. The process for preparing γ-polyglutamic acid from high-viscous culture broth of claim 1, wherein the culturing is performed in a culture medium containing 60-100 g/L glycerol, 10-30 g/L L-glutamic acid, 5-20 g/L citric acid, 3-10 g/L NH₄Cl, 0.1-1.0 g/L K₂HPO₄, 0.1-2.0 g/L MgSO₄. 7H₂O, 0.01-0.1 g/L FeCl₃.6H₂O, 0.05-0.5 g/L CaCl₂.2H₂O and 0.05-0.5 g/L MnSO₄.H₂O.

4. The process for preparing γ-polyglutamic acid from high-viscous culture broth of claim 1, wherein the membrane is an ultrafiltration membrane with exclusion molecular weight of 50-100 kDa or a filtration membrane with pore size of 0.15-0.5 μm.

5. The process for preparing γ-polyglutamic acid from high-viscous culture broth of claim 1, wherein ethanol is added to the concentrate at a volume ratio of 4:1-2:1.
